SEQUENCE LISTING (22 sequences)

SEQ ID NO: 1 - protein, 10 aa, Homo sapiens
CGGSGVEDLS

SEQ ID NO: 2 - protein, 7 aa, Homo sapiens
RLPSGEE

SEQ ID NO: 3 - protein, 6 aa, Homo sapiens
NITEGE

SEQ ID NO: 4 - protein, 7 aa, Homo sapiens
ARGSVIL

SEQ ID NO: 5 - protein, 4 aa, Homo sapiens
EDLS

SEQ ID NO: 6 - protein, 5 aa, Homo sapiens
VEDLS

SEQ ID NO: 7 - protein, 6 aa, Homo sapiens
GVEDLS

SEQ ID NO: 8 - protein, 7 aa, Homo sapiens
SGVEDLS

SEQ ID NO: 9 - protein, 8 aa, Homo sapiens
GSGVEDLS

SEQ ID NO: 10 - protein, 9 aa, Homo sapiens
GGSGVEDLS

SEQ ID NO: 11 - protein, 4 aa, Homo sapiens
RLPS

SEQ ID NO: 12 - protein, 5 aa, Homo sapiens
RLPSG

SEQ ID NO: 13 - protein, 6 aa, Homo sapiens
RLPSGE

SEQ ID NO: 14 - protein, 6 aa, Artificial
  Aggrecan synthetic peptide
  PEPTIDE (1)..(6)
DLSRLP

SEQ ID NO: 15 - protein, 7 aa, Artificial
  Aggrecan synthetic peptide
  PEPTIDE (1)..(7)
DLSRLPS

SEQ ID NO: 16 - protein, 7 aa, Artificial
  Aggrecan synthetic peptide
  PEPTIDE (1)..(7)
EDLSRLP

SEQ ID NO: 17 - protein, 8 aa, Artificial
  Aggrecan synthetic peptide
  PEPTIDE (1)..(8)
DLSRLPSG

SEQ ID NO: 18 - protein, 8 aa, Artificial
  Aggrecan synthetic peptide
  PEPTIDE (1)..(8)
VEDLSRLP

SEQ ID NO: 19 - protein, 9 aa, Artificial
  Aggrecan synthetic peptide
  PEPTIDE (1)..(9)
DLSRLPSGE

SEQ ID NO: 20 - protein, 9 aa, Artificial
  Aggrecan synthetic peptide
  PEPTIDE (1)..(9)
GVEDLSRLP

SEQ ID NO: 21 - protein, 4 aa, Artificial
  Aggrecan synthetic peptide with conservative variant amino acid substitution
  PEPTIDE (1)..(4)
DDLS

SEQ ID NO: 22 - protein, 4 aa, Artificial
  Aggrecan synthetic peptide with conservative variant amino acid substitution
  PEPTIDE (1)..(4)
EELS

(19) United States
(12) Patent Application Publication
(10) Pub. No.: US 2007/0099246 A1
(43) Pub. Date: May 3, 2007

(54) Antibodies, assays and kits to quantitate cartilage destruction

(76) Inventors: John D. Sandy, Tampa, FL (US); Koichi Masuda, Wilmette, IL (US); Brian Pfister, Wilmette, IL (US)

Correspondence Address: LAWSON & WEITZEN, LLP, 88 BLACK FALCON AVE, SUITE 345, BOSTON, MA, 02210, US

(21) Appl. No.: 11/591,307

(22) Filed: Nov. 1, 2006

Related U.S. Application Data
(60) Provisional application No. 60/732,976, filed on Nov. 3, 2005.

(51) Int. Cl. G01N 33/567; C12P 21/04; C07K 16/40

(52) U.S. Cl. 435/7.2; 530/388.26; 435/70.21

(57) ABSTRACT

Antibody compositions specific for peptides released during arthritic cartilage destruction are provided as reagents for assay of progression of arthritic processes for antibodies, and as diagnostic and prognostic agents. Also provided are methods and kits for this assay.

### RELATED APPLICATIONS

The present application claims the benefit of provisional application Ser. No. 60/732,976 filed in the U.S. Patent and Trademark Office on Nov. 3, 2005, which is hereby incorporated by reference herein in its entirety.

### TECHNICAL FIELD

The present invention relates to antibody compositions, assays and kits to quantitate cartilage destruction.

### BACKGROUND

Arthritic diseases are ubiquitous in an aging population, with costs in 1991 of knee replacements alone due to osteoarthritis exceeding one billion dollars. Many current methods for determining presence and rate of cartilage destruction during arthritic processes are invasive, or are associated with highly technological and expensive equipment and are performed only at specialized centers. These methods include digital imaging analysis of microscopic images of biopsied tissue in experimental animals, and for human patients, include CAT scans (CT), magnetic resonance imaging (MR), and ultrasound.

While assays of circulating markers for cartilage destruction have been described, none have identified a product generated during destructive processes, rather than by normal turnover processes such as has been found by immunochemical analysis of type II collagen degradation (Dodge et al., J Clin Invest 1989 83: 647-672). Improved convenient and rapid assays and reagents for assays that are specific for detecting cartilage destruction are needed.

### SUMMARY

An embodiment of the invention herein provides an antibody that binds to a target ligand including a calpain-generated epitope, such that aggrecanase-mediated degradation of aggrecan releases the target from cartilage in a tissue of a subject. In a particular embodiment, the calpain is m-calpain. Further, the target has a carboxy-terminal amino acid sequence having aspartic acid-leucine-serine (DLS). In a related embodiment, the antibody target has an amino-terminal having the sequence alanine-arginine-glycine (ARG). In another related embodiment, the target ligand is soluble in body fluids.

The antibody in various embodiments can be a plurality of antibodies, for example, a composition having a combination of antibodies, each component antibody binding to a different target epitope, or to the same epitope.

Thus the target detected by the antibody of the invention herein in a sample of body fluid is further characterized as aggrecan peptide Ala393-Ser1411, and the term, “target” as used herein refers specifically to this peptide. See Sandy et al., Biochem J. 2001 Sep. 15: 358: 615-626. The target is generated by proteolytic cleavage mediated by m-calpain between amino acid residues at positions 1411 and 1412 of the aggrecan target peptide. m-Calpain digestion produces two “neo-epitopes” as follows. At the C-terminus of the target peptide, the amino acid sequence of ten positions, 1402-1411, is CGGSGVEDLS (SEQ ID NO: 1, using the conventional one letter abbreviations for the amino acids). At the N-terminus of the remainder of aggrecan starting with amino acid position 1412 is the sequence RLPSGEE (SEQ ID NO: 2).

The numbers of the residue positions refer to the system of numbering in human aggrecan, however similar or identical residue positions can be identified in other mammals. The enzyme aggrecanase digests aggrecan between residues at amino acid positions 392 and 393, generating ends having the following amino acid sequences: NITEGE (SEQ ID NO: 3) which are positions 387-392 at the C-terminus of the fragment 1-392, and ARGSVIL (SEQ ID NO: 4) at positions 393-399 at the amino terminus of the target peptide.

Accordingly, the invention herein provides the epitopes for production of antibodies for detection of the target peptide and for other fragments of aggrecan: CGGSGVEDLS (SEQ ID NO: 1); RLPSGEE (SEQ ID NO: 2); EDLS (SEQ ID NO: 5); VEDLS (SEQ ID NO: 6); GVEDLS (SEQ ID NO: 7); SGVEDLS (SEQ ID NO: 8); GSGVEDLS (SEQ ID NO: 9); GGSGVEDLS (SEQ ID NO: 10); RLPS (SEQ ID NO: 11); RLPSG (SEQ ID NO: 12); and, RLPSGE (SEQ ID NO: 13).

Further, treatment of a sample containing the target with trypsin, chondroitinase and neuraminidase, is a digestion that results in the appearance of a peptide Ileu925-Ser1141 having disaccharide substitutions, that complexes with the antibody herein to make a “sandwich” which can be detected with peanut agglutinin peroxidase, or can be biotinylated before binding and detected with avidin peroxidase, or is detected with one or more monoclonal antibodies (Mabs) to chondroitin sulfate (CS)-stubs Glant et al., J. Immunol 1998 106: 3812-3819), or detecting keratan sulfate (KS) chains (Arth. Rheum. 1998 41(6): 1019-1025) or epitopes of other protein such as, using the one letter code for amino acids, anti-ARGSV which is a portion of SEQ ID NO: 4 (Sandy et al., Ibid.), anti-G2 domain (Roughley et al., 2003 Biochem J. 375: 183-189), or anti-CS1 domain, all of which are well known methods in the art of assay, and all of which use commercially available reagents. An antibody produced to any of the epitopes on the target as defined herein containing the sequence DLS such as antibody produced with any of SEQ ID NOs: 1 and 5-10, is useful to detect this target peptide derivative. Further, combinations of this antibody with other antibodies having the same or different specific binding affinities are provided.

In general, the antibody binds the ligand, which is an epitope located on that portion of a cartilage target protein which is soluble in body fluids. The ligand appears in body fluids as a result of the degradation of the cartilage, after an appropriate lag time.

The antibody in various embodiments is selected from the group of antibodies consisting of a polyclonal, monoclonal, Fab, single chain antibody (scAb) and an antigen-binding determinant of a heavy chain and a light chain. In general, the antibody is polyclonal, however monoclonal antibodies and others are within the scope of the invention. The antibody is an IgG, such as IgG1 or IgG4, however other types such as IgA, IgE and IgM are envisioned as useful herein.

The antibody is generally produced by contacting an animal with an immunogen or an epitope, which is generally a peptide having an amino acid sequence, using the convention three letter amino acid abbreviations: cys-gly-gly-ser-gly-val-glu-asp-leu-ser (SEQ ID NO: 1), glu-asp-leu-ser (SEQ ID NO: 5), val-glu-asp-leu-ser (SEQ ID NO: 6), gly-val-glu-asp-leu-ser (SEQ ID NO: 7), ser-gly-val-glu-asp-leu-ser (SEQ ID NO: 8), gly-ser-gly-val-glu-asp-leu-ser (SEQ ID NO: 9), and gly-gly-ser-gly-val-glu-asp-leu-ser (SEQ ID NO: 10), and conservative variants of these sequences. Conservative variants such as amino acid substitutions are well known in the art, and examples are provided infra.

The target recognized by the antibody described herein is an in vivo degradation product of stable matrix aggrecan. Particularly in a subject having an arthritic condition, newly synthesized cartilage is cleaved at each of the N-terminal and C-terminal of aggrecan by aggrecanase, to generate peptide Ala393-Gly1564. Further, it is observed that when aggrecanase attacks the stable intercellular matrix, it generates both Ala393-Glu1564 and the target peptide of the antibody herein, which is peptide Ala393-Ser1411. The target peptide Ala393-Ser1411 is generated as a result of aggrecanase mediated cleavage of a pre-existing calpain cleavage product, as shown in FIG. 1 herein.

An embodiment of the invention herein provides a method of making the antibody above, the method including: contacting an animal with a synthetic peptide having an amino acid sequence that is an calpain substrate, such that the animal produces the antibody; and obtaining the antibody from the serum of the animal. Generally in the method, the animal is selected from the group of rabbit; mouse; rat; dog; horse; cow; sheep; pig; and goat, although any mammal preferentially, or a bird or reptile would be suitable for producing the antibody. The method further involves contacting the animal with the amino acid sequence shown in SEQ ID NO: 1 or SEQ ID NOs: 5-10, or a conservative variant of any of these. In other embodiments, the method further involves contacting the animal with the amino acid sequence selected from at least of the group consisting of asp-leu-ser (DLS), arg-leu-pro (RLP), and conservative variants of these sequences. “Contacting the animal” as used herein means contacting the immune system by administering the amino acid sequence shown in SEQ ID NO: 1 or SEQ ID NOs: 5-10 parenterally to the animal by means well known in the art, for example, in an emulsion with Freund's adjuvant. Parenteral administration is by injection, e.g., intravenous, subcutaneous, or intraperitoneal, and additional parenteral routes are within the scope of the invention.

Similarly in additional embodiments, the invention provides a method that further involves contacting the animal with the amino acid sequence shown in SEQ ID NO: 2 or any of SEQ ID NOs: 11-13, or a conservative variant of any of these, alone or in a combination of two or more.

Also provided herein is a method of assaying cartilage destruction in a subject including contacting a sample of a body fluid of the subject with any antibody described herein specific for the target; and determining a presence of the epitope having amino acids aspartic acid-leucine-serine (DLS as is found in any of SEQ ID NOs: 1 and 5-10) or arg-leu-pro (RLP) in the fluid that binds to the antibody, such that the amount of the epitope is a measure of cartilage destruction in the subject.

The assay in general is useful for diagnosing a subject who is at risk for having an arthritic condition, or a subject having an arthritic condition, or for prognosing the course of an arthritic condition, however the assay is useful also for diagnosing normal subjects who might be at risk for an arthritic condition. The arthritic condition is at least one selected from group of rheumatoid arthritis, osteoarthritis, and juvenile arthritis, traumatic injury, ankylosing spondylitis, infectious arthritis, psoriatic arthritis, lumbosacral arthritis, systemic lupus erythematosus (SLE), degenerative disc disease, gout, pseudogout, and reactive arthritis, and related conditions. In an embodiment, the antibody is a polyclonal antibody. Alternatively, the antibody is a monoclonal antibody, and combinations of various antibodies are within the scope of the invention.

The assay can be a well-known “sandwich” assay, in which the antibody or at least a primary antibody is immobilized. In an embodiment of the sandwich assay, the epitope is detectably labeled, for example, the labeled epitope is at least one selected from radioactive, chemiluminescent, bioluminescent, and colorimetric. In a particular related embodiment, the method further involves, prior to determining, biotinylating the epitope. Alternatively, the method further involves, prior to determining, detecting the epitope by treating the sample with trypsin, chondroitinase and neuraminidase to obtain a resulting disaccharide-substituted Ileu925-Ser1411 derivative of the target. In a related embodiment, the method further involves, prior to determining, deglycosylating the sample with an enzyme selected from at least of the group of chondroitinase ABC, chondroitinase A, chondroitinase B, chondroitinase C, chondroitinase AC, keratanase, and keratanase II. Further, the method involves contacting a complex of antibody and resulting derivative of the target with peanut agglutinin peroxidase. Alternative detection systems are described above, and include detecting the sandwich with Mabs (secondary antibodies) to CS-stubs, KS chains, anti-ARGSV as found in a portion of SEQ ID NO: 4, anti-G2 domains, or anti-CS1 domains.

In various embodiments, the body fluid sample from the subject selected from the group consisting of blood, urine, synovial fluid, tears, sweat, saliva, serum, lymph, semen, vaginal fluid, cerebro-spinal fluid, cell culture supernatant, cell extract, and tissue extract. In a related embodiment, the method further involves, prior to contacting, providing the sample from the subject from at least one selected from the group consisting of urine, synovial fluid, serum, and tissue extract. Blood, serum, urine, synovial fluid, and tissue extract, are useful for applying the method as a tool in pre-clinical stages of research for development of the antibody for diagnostics or therapeutics.

The method involves the antibody or a combination, referred to as “primary antibodies”, immobilized on a support substrate. The support substrate is selected from the group of a bead, a slide, a gel, a multi-well plate, and a column. The method further, in various related embodiments, involves comparing the amount of epitope in the sample to a control lacking the epitope.

In another embodiment, the method further involves, prior to contacting, administering to the subject an agent for treating a cartilage destruction condition, such that the amount of epitope is a measure of prevention of progressive cartilage destruction.

Another aspect of the invention provides a method of assaying for prevention or amelioration of progressive cartilage destruction in a subject. The method includes administering to the subject an agent for treating a cartilage destruction condition; contacting at least one sample of a body fluid of the subject with at least one antibody described above; and determining a presence of at least one epitope having amino acid sequences aspartic acid leucine-serine (DLS) or arg-leu-pro (RLP) in the fluid, such that the epitope binds to the antibody, and the amount of the epitope that binds is a measure of cartilage destruction or prevention of destruction in the subject, compared to a control sample of the body fluid obtained prior to administering, or to a control subject not administered the agent.

A further aspect of the invention provides a method for assaying activity of m-calpain in a sample in need of an assay. The method involves contacting the sample with an m-calpain substrate to produce a resulting reaction mix, such that the substrate is aggrecan, or a synthetic peptide having an amino acid sequence of an m-calpain digestion site; and determining a presence and an amount of m-calpain, by reacting the reaction mix with at least one antibody specific for a neo-epitope resulting from m-calpain digestion of the aggrecan, such that the extent of the reaction of the sample with the at least one antibody, in comparison to a control reaction in the absence of the sample, is an indication of the presence and the amount of m-calpain.

In a related embodiment of the method, the synthetic peptide includes an amino acid sequence asp-leu-ser-arg-leu-pro (DLSRLP; SEQ ID NO: 14). In another related embodiment of the method, the synthetic peptide includes an amino acid sequence selected from the group: asp-leu-ser-arg-leu-pro-ser (DLSRLPS; SEQ ID NO: 15); glu-asp-leu-ser-arg-leu-pro (EDLSRLP; SEQ ID NO: 16); asp-leu-ser-arg-leu-pro-ser-gly (DLSRLPSG; SEQ ID NO: 17); val-glu-asp-leu-ser-arg-leu-pro (VEDLSRLP; SEQ ID NO: 18); asp-leu-ser-arg-leu-pro-ser-gly-glu (DLSRLPSGE; SEQ ID NO: 19); and gly-val-glu-asp-leu-ser-arg-leu-pro (GVEDLSRLP; SEQ ID NO: 20).

Another aspect of the invention provides a method for detecting in a sample of a body fluid from a subject, an auto-antibody bound to an aggrecan peptide. The method involves immobilizing on a solid surface at least one antibody produced by immunizing an animal with a synthetic peptide having amino acid sequence as described above; contacting the surface with the fluid, such that the aggrecan peptide and autoantibody bind to the surface; and detecting the autoantibody with a reagent that binds to at least one Fc constant amino acid sequence for the subject species, such that the auto-antibody is detected in the sample.

In a related embodiment of the method, the subject is a human. In another related embodiment of the method, the subject is identified as having or being at risk for an autoimmune disease. In a particular embodiment, the autoimmune disease is rheumatoid arthritis.

Yet another embodiment provided herein is a kit including an antibody, or at least one antibody according to any of those described above, for assaying cartilage destruction in a subject, such that the antibody is present in a unit dose. The term “dose” as used herein refers to a precise amount of an antibody or a combination of antibodies, present in the kit, that amount having been determined to be useful for a functional assay. Generally the kit contains a plurality of doses, for example, the kit for example has the correct “dose” of at least one antibody immobilized in each of a plurality of wells in a multi-well plate. The kit further contains instructions for use, and in various embodiments, at least one container. Alternatively or additionally, the kit further contains a positive control, for example, the positive control is an amount of the aggrecan target ligand.

### DETAILED DESCRIPTION OF EMBODIMENTS

An object of the invention herein is to provide a method which is an efficient assay to quantitate cartilage destruction. Accordingly, an embodiment of the invention provides a novel antibody that binds to a target ligand having an epitope produced from degradation of aggrecan in cartilage. The target ligand of the antibody, or “target”, arises during destruction of the cartilage by degradation that is the result of activities of endogenous proteases, calpain and aggrecanase. The target has a carboxy-terminal amino acid sequence including aspartic acid-leucine-serine (DLS). Further, the target ligand is soluble in body fluids, as compared to healthy cartilage which is a structural material found in joints of the skeletal system. Further as a result of the degradation of cartilage, the target has an amino-terminal having ala-arginine-glycine (ARG). Further, an embodiment of the target includes amino acids having the sequence of positions 393-1411 of human aggrecan.

In various embodiments of the invention, the antibody above is selected from the group of antibodies consisting of a polyclonal, monoclonal, Fab, single chain antibody (scAb) and an antigen-binding determinant of a heavy chain and a light chain. In general, the antibody is polyclonal or monoclonal. In general, the antibody is an IgG, although other isotypes are possible, for example, IgA, or IgM. Further, the antibody does not detect the epitope DLS in cartilage in situ, because the sample of the body fluid does not contain structural cartilage, rather the body fluid sample gives a snap shot of the breakdown products of cartilage, among which is the target peptide ligand of the antibodies herein.

The antibody which is polyclonal is produced by contacting an animal with a peptide having an amino acid sequence cys-gly-gly-ser-gly-val-glu-asp-leu-ser (SEQ ID NO: 1), or a conservative variant of these. This amino acid sequence contains the signature DLS tripeptide fragment characteristic of the epitope, and is a convenient immunogen, as it can be prepared synthetically by standard methods of amino acid synthesis. A monoclonal antibody is made using antibody-producing lymphocytes obtained from an animal so immunized, for fusion to immortalize the cells, by techniques well known to one of ordinary skill in the art of immunology.

In yet another embodiment, the invention provides a method of making an antibody as described above, the method having steps of: contacting an animal with a peptide having an amino acid sequence that is or contains an m-calpain target, such that the animal produces the antibody specific for binding to the amino acid sequence; and obtaining the antibody from the serum of the animal.

In general, the animal used for immunization is a mammal, for example, is selected from the group of rabbit; mouse; rat; dog; horse; cow; sheep; pig; and goat. Other animals capable of producing antibodies in response to the peptide are within the scope of the invention, for example, antibodies can be prepared using a reptile or a bird.

Yet another embodiment of the invention provides a method of assaying cartilage destruction in a subject including contacting a sample of a body fluid of the subject with an antibody as described above; and determining an amount of epitope having amino acids aspartic acid leucine-serine (DLS) in the fluid that binds to the antibody, such that the amount of the epitope is a measure of cartilage destruction in the subject.

In general, the antibody used in the method is a polyclonal antibody. Alternatively, the antibody is a monoclonal antibody or a combination of monoclonal antibodies or a combination of polyclonal antibodies. In an embodiment of the method, the antibody is immobilized, for example, the antibody is immobilized on a support substrate selected from the group of a bead, a slide, a gel, a multi-well plate, and a column. Combinations of antibodies can be made for binding to each neo-epitope in the target peptide, or can be made for recognizing more than one degradation product.

In embodiments of the method, the epitope is detectably labeled, for example, the labeled epitope is selected from radioactive, chemiluminescent, bioluminescent, and colorimetric. In a further embodiment, the method further involves, prior to contacting, biotinylating the epitope. In various further embodiments, the method further involves prior to contacting, providing a sample from the subject selected from the group consisting of blood, urine, synovial fluid, tears, sweat, saliva, serum, lymph, semen, vaginal fluid, cerebro-spinal fluid, cell culture supernatant, cell extract, and tissue extract. The method in various embodiments can further include comparing the amount of epitope in the sample to a control lacking the epitope, i.e., a negative control. In an alternative embodiment, the method can further include comparing the amount of epitope in the sample to a control having a known amount of the epitope, i.e., a positive control.

Also provided herein is a kit including an antibody according to any of above, for assaying cartilage destruction in a subject, for example, in a body fluid sample taken from a subject, in a container. In general, the antibody is present in a unit dose, e.g., is present in a plurality of unit doses. Thus, the antibody is provided in an immobilized form in a plurality of wells in a multi-well plate. The kit can further include other items, such as buffers, instructions for use, and a positive control, for example, the positive control is an amount of the aggrecan target peptide ligand.

The methods of use and kits in various embodiments are used to assay cartilage degradation, to determine if a subject has or is at risk of having an arthritic condition. The term, “arthritic conditions” includes rheumatoid arthritis, osteo-arthritis, and juvenile arthritis, and also includes without limitation other conditions such as joint pain.

### BRIEF DESCRIPTION OF THE DRAWINGS

FIG. 1 is a drawing showing aggrecan fragments generated by enzymatic cleavage.

FIG. 1 panel A shows aggrecan protein in cartilage, having a length of 2415 amino acids.

FIG. 1 panel B shows aggrecan of length 1411 following digestion by m-calpain between serine at position 1411 and position 1412 during processing in normal articular cartilage. The new C-terminus after digestion by m-calpain is Asp-Leu-Ser (DLS) and is a neo-epitope provided herein.

FIG. 1 panel C shows aggrecan released from cartilage following aggrecanase activity, which cleaves between glutamic acid at 392 and alanine at 393. The new N-terminus of the target polypeptide released from aggrecan, which extends from position 393 to 1411, is indicated ARG in the figure, and refers to the sequence of amino acids Ala-Arg-Gly that are found in positions 393 to 395.

### DEFINITIONS

In order that the present invention may be more readily understood, certain terms are defined, with additional definitions set forth throughout the description. As used in the specification and in the claims herein, the following words and phrases have the meaning below.

The term “aggrecan” means a proteoglyean or fragment of a proteoglycan present in articular cartilage. The molecular weight of aggrecan in the tissue ranges from 1 to 3.5 MDa, and the size as determined by electron microscopy varies in the range of 100-300 nm. Aggrecan monomers contain two extended regions which carry the bulk of the glycosaminogly can and three globular domains, G1 and G2 at the N-terminus and G3 at the C-terminus of the core protein. The C-terminal G3 domain includes an alternatively spliced complement regulatory protein-like repeat at the extreme C-terminus, an adjacent repeat homologous with C-type animal lectins and an N-terminal epidermal growth factor (EGF)-like domain that is also subject to alternative splicing.

Aggrecan core protein in articular cartilage is present as multiple species generated by C-terminal truncation of the full-length protein. Aggrecan is abundant in normal human articular cartilage and is released into body fluids when cleaved by enzymes, for example, aggrecanase. In joint pathology, aggrecan loss leads to progressive cartilage degradation and results in arthritic conditions, including for example, osteoarthritis (OA) or rheumatoid arthritis (RA). Release of fragments of aggrecan and several of its epitopes in increased amounts during the course of these pathological conditions, as shown herein, enables measurement to be made in synovial fluid and serum, and even in urine.

The term “aggrecanase” means a multidomain protein consisting of prodomains, protease, disintegrin, thrombospondin and cys-rich domains followed by spacer regions and additional thrombospondin motifs. The prodomains are cleaved-off intracellularly and the enzymes are processed further from their C-terminal ends after secretion. Aggrecanase is a member of a disintegrin and metalloprotease protein families, with thrombospondin motifs (ADAMTS). Aggrecanase cleaves specific peptide bonds in aggrecan and hydrolyzes other lecticans, such as, as versican and brevican. Aggrecanase is an enzyme associated with excessive aggrecan degradation.

The term “m-calpain” means a heterodimeric calcium-dependent cysteine protease consisting of catalytic and regulatory subunits. The effects of calcium on the enzyme include activation, autolysis, and subunit dissociation. M-calpain is in the family of matrix metalloproteinases (MMPs) and is involved in the baseline turnover of aggrecan.

Aggrecan is the major space-filling proteoglycan present in articular cartilage, i.e., cartilage found in joints. Aggrecan provides the tissue with mechanical properties of reversible compressibility. In joint pathology, such as that seen in osteoarthritis (OA) and rheumatoid arthritis (RA), aggrecan loss leads to progressive cartilage degradation.

It is now well established that aggrecan core protein in mature articular cartilages is present as multiple species generated by C-terminal truncation of the full-length protein. Despite intensive study of these C-terminally truncated species and attempts to identify the proteinases responsible for their generation in vivo, the precise structure has been established only for two of the five major separable forms. Forms of cartilage that have been identified are generated by activity of the enzyme aggrecanase (the product known as G1-NITEGE392) and a metalloproteinase (MMP) activity (the product known as G1-VDIPEN360). It is generally accepted that the aggrecanase activity is responsible for matrix degradation in human disease.

Without being limited by any particular theory or mechanism of action, various embodiments of the invention herein arise from the discovery that a precise C-terminal sequence of a major aggrecan species (arising when aggrecan is cleaved by m-calpain) can be accurately assayed from a sample such that the amount of the product species correlates with amount of cartilage destruction. Further, various embodiments of the invention herein provide a new polyclonal antibody (called JSCDLS) that has the capacity to bind to and therefore to detect and quantify fragments of aggrecan in human body fluids.

These fragments are generated by aggrecanase activity when the enzyme cleaves the m-calpain-cleaved cartilage aggrecan. The m-calpain-cleaved cartilage aggrecan represents the stable, long-lived and a structurally critical component of the cartilage aggrecan matrix. Aggrecanase also cleaves the newly synthesized population of aggrecan molecules, however cleavage of the newly synthesized population of aggrecan molecules does not result in fragments that react with the embodiment of the invention that is a method of assay using the antibody described herein (JSCDLS).

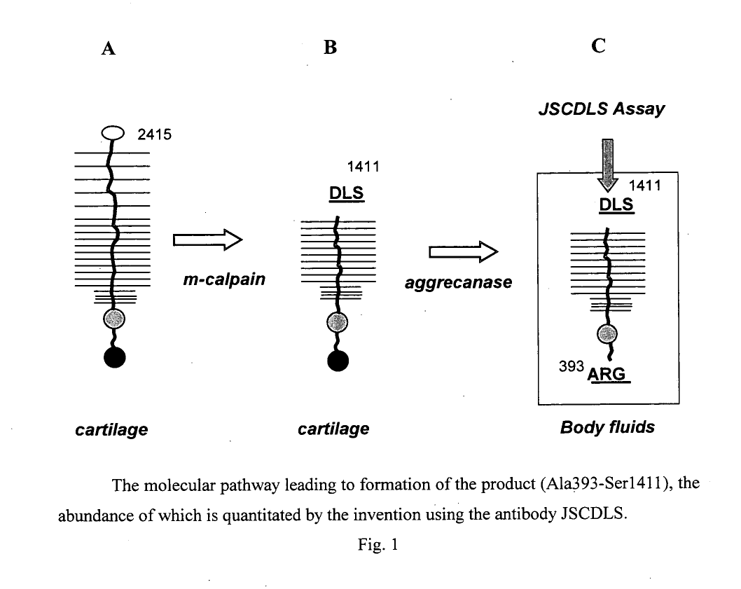

The subject assay uses the antibody that specifically binds to, and therefore detects aggrecan that has been cleaved by m-calpain at a specific site (serine 1411-arginine 1412), in the human aggrecan CS-1 domain (see FIG. 1B).

Aggrecan bearing a C-terminal amino acid sequence Asp-Leu-Ser (DLS) is abundant in normal human articular cartilage and, most importantly, it is released into body fluids when aggrecanase cleaves at a site between the positions of two adjacent amino acid residues, at Glu392-Ala393 in human aggrecan.

Release into body fluids of the DLS-containing fragment provides a soluble epitope that represents a molecular signal for destruction of the stable mature aggrecan matrix, and this signal is a direct measure of advancing or progressive human arthritis. This distinguishes the assay from other aggrecan fragment assays that detect fragments generated by normal turnover processes and in early disease only.

The DLS assay is based on the ELISA technique in which the antibody (referred to in the figure as JSCDLS), or a combination of a plurality of different antibodies, is coated on multiwell plates and the aggrecan fragments (or products derived from these fragments) are biotinylated and captured by the antibody. The specifically captured biotinylated aggrecan fragments, which are representatives of the target peptide ligand as defined herein, are then quantitated, for example, by binding of avidin peroxidase and generating peroxidase products using standard methods well known to one of ordinary skill in the art of immunoassays. The readout is generally a colorimetric measurement that is suitable for use in a doctor's office, and is non-radioactive, rapid, and suitable for scale-up using a large number of multi-well dishes.

Antibodies

The present invention relates to isolated antibodies, particularly human antibodies, that bind specifically to the epitope referred to herein as “target”, which is a peptide degradation product of aggrecan that is released during cartilage degradation. The invention provides antibodies, including isolated antibodies, methods of making such antibodies, immunoconjugates and bispecific molecules including such antibodies and pharmaceutical compositions containing the antibodies, immunoconjugates or bispecific molecules of the invention. The invention also relates to methods of using the antibodies to assay for cartilage degradation, for diagnosis or prognosis of an arthritic condition.

The term “antibody” as referred to herein includes whole antibodies and any antigen binding fragment (i.e., “antigen-binding portion”) or single chains of these. A naturally occurring “antibody” is a glycoprotein including at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full-length or one or more fragments of an antibody that retain the ability to specifically bind to an antigen (e.g., the target peptide fragment of aggrecan as defined herein). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds to the target, and is substantially free of antibodies that specifically bind antigens other than the target). An isolated antibody that specifically binds the target may, however, have cross-reactivity to other antigens, such as the target molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. As the target is of human origin, in general these antibodies are made using a non-human animal, or an in vitro method such as phage display. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM, IgE, IgG such as IgG1 or IgG4) that is provided by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human target” is intended to refer to an antibody that binds to human target with a K_{D} of 5×10⁻⁹ M or less, 2×10⁻⁹ M or less, or 1×10⁻¹⁰ M or less. An antibody that “cross-reacts with an antigen other than human target” is intended to refer to an antibody that binds that antigen with a K_{D} of 0.5×10⁻⁸ M or less, 5×10⁻⁹ M or less, or 2×10⁻⁹ M or less. An antibody that “does not cross-react with a particular antigen” is intended to refer to an antibody that binds to that antigen, with a K_{D} of 1.5×10⁻⁸ M or greater, or a K_{D} of 5-10×10⁻⁸ M or 1×10⁻⁷ M or greater. In certain embodiments, such antibodies that do not cross-react with the antigen exhibit essentially undetectable binding against these proteins in standard binding assays.

The term “K_{assoc}” or “Kₐ”, as used herein, is intended to refer to the association rate of a particular antibody-antigen interaction, whereas the term “K_{dis}” or “K_{D},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

In order to get a higher avidity probe, a dimeric conjugate (two molecules of the antibody coupled to a FACS marker) can be constructed, thus making low affinity interactions (such as with the germline antibody) more readily detected by FACS. In addition, another means to increase the avidity of antigen binding involves generating dimers or multimers of any of constructs encoding antibodies as described herein. Such multimers may be generated through covalent binding between individual modules, for example, by imitating the natural C-to-N-terminus binding or by imitating antibody dimers that are held together through their constant regions. The bonds engineered into the Fc/Fc interface may be covalent or non-covalent. In addition, dimerizing or multimerizing partners other than Fc can be used in the antibody hybrids to create such higher order structures.

As used herein, the term “cross-reactivity” refers to an antibody or population of antibodies binding to epitopes on other antigens. This can be caused either by low avidity or specificity of the antibody or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “high affinity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, or 10⁻⁸ M or less.

As used herein, the term “subject” includes any human or nonhuman animal. The term “nonhuman animal” includes all vertebrates, e.g., mammals and non-mammals, all warm blooded vertebrates such as mammals and birds, nonhuman primates, sheep, dogs, cats, horses, cows chickens, amphibians, reptiles, etc.

Standard assays to evaluate the binding ability of the antibodies toward the target of various species are known in the art, including for example, ELISAs, western blots and RIAs. Suitable assays are described in detail in the Examples. The binding kinetics (e.g., binding affinity) of the antibodies also can be assessed by standard assays known in the art, such as by Biacore analysis. Assays to evaluate the effects of the antibodies on functional properties of the target (e.g., receptor binding, preventing or ameliorating osteolysis) are described in further detail in the Examples.

Monoclonal Antibodies

Since each of these antibodies can bind to the target, the V_{H}, V_{L}, full-length light chain, and full-length heavy chain sequences (nucleotide sequences and amino acid sequences) can be “mixed and matched” to create other anti-target binding molecules of the invention. The target binding of such “mixed and matched” antibodies can be tested using the binding assays described above and in the Examples (e.g., ELISAs). When these chains are mixed and matched, a V_{H} sequence from a particular V_{H}/V_{L} pairing should be replaced with a structurally similar V_{H} sequence. Likewise a full-length heavy chain sequence from a particular full-length heavy chain/full-length light chain pairing should be replaced with a structurally similar full-length heavy chain sequence. Likewise, a V_{L} sequence from a particular V_{H}/V_{L} pairing should be replaced with a structurally similar V_{L} sequence. Likewise a full-length light chain sequence from a particular full-length heavy chain/full-length light chain pairing should be replaced with a structurally similar full-length light chain sequence. The V_{H}, V_{L}, full-length light chain, and full-length heavy chain sequences of the antibodies of the present invention are particularly amenable for mixing and matching, since these antibodies use V_{H}, V_{L}, full-length light chain, and full-length heavy chain sequences derived from the same germline sequences and thus exhibit structural similarity.

Given that each of the antibodies can bind to the target and that antigen-binding specificity is provided primarily by the CDR1, 2 and 3 regions, the V_{H} CDR1, 2 and 3 sequences and V_{L} CDR1, 2 and 3 sequences can be “mixed and matched” (i.e., CDRs from different antibodies can be mixed and match, although each antibody must contain a V_{H} CDR1, 2 and 3 and a V_{L} CDR1, 2 and 3 to create other anti-target antibodies and/or binding molecules of the invention. The target binding of such “mixed and matched” antibodies can be tested using the binding assays described above and in the Examples (e.g., ELISAs). When V_{H} CDR sequences are mixed and matched, the CDR1, CDR2 and/or CDR3 sequence from a particular V_{H} sequence should be replaced with a structurally similar CDR sequence(s). Likewise, when V_{L} CDR sequences are mixed and matched, the CDR1, CDR2 and/or CDR3 sequence from a particular V_{L} sequence should be replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences shown herein for monoclonal antibodies of the present invention.

As used herein, a human antibody comprises heavy or light chain variable regions or full-length heavy or light chains that are “the product of” or “derived from” a particular germline sequence if the variable regions or full-length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a selected human antibody typically is at least 90% identical in amino acids sequence to an amino acid sequence encoded by a germline immunoglobulin gene and contains amino acid residues that identify the antibody as being human or appropriate to some other animal of origin, when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, antibody derived from a particular germline sequence will display no more than 10 amino acid differences from the amino acid sequence encoded by the germline immunoglobulin gene. In certain cases, the antibody may display no more than 5, or even no more than 4, 3, 2, or 1 amino acid difference from the amino acid sequence encoded by the germline immunoglobulin gene.

Homologous Antibodies

In yet another embodiment, an antibody of the invention has full-length heavy and light chain amino acid sequences; full-length heavy and light chain nucleotide sequences, variable region heavy and light chain nucleotide sequences, or variable region heavy and light chain amino acid sequences that are homologous to the amino acid and nucleotide sequences of the antibodies described herein, and such that the antibodies retain the desired functional properties of the anti-target antibodies of the Invention.

For example, the invention provides an isolated monoclonal antibody, or antigen binding portion of these, comprising a heavy chain variable region and a light chain variable region, wherein: the heavy chain variable region comprises an amino acid sequence that is at least 80% homologous to an amino acid sequence; the light chain variable region comprises an amino acid sequence that is at least 80% homologous to an amino acid sequence; and the antibody specifically binds to the target.

The antibody can be, for example, a human antibody, a humanized antibody or a chimeric antibody. In other embodiments, the V_{H} and/or V_{L} amino acid sequences may be 50%, 60%, 70%, 80%, 90%, 95%, 96%, 97%, 98% or 99% homologous to sequences that are designated. An antibody having V_{H} and V_{L} regions having high (i.e., 80% or greater) homology to the V_{H} and V_{L} regions, can be obtained by mutagenesis (e.g., site-directed or PCR-mediated mutagenesis) of nucleic acid molecules, followed by testing of the encoded altered antibody for retained function (i.e., the functions set forth above) using the functional assays described herein.

In other embodiments, the full-length heavy chain and/or full-length light chain amino acid sequences may be 50% 60%, 70%, 80%, 90%, 95%, 96%, 97%, 98% or 99% homologous to the sequences set forth above. In other embodiments, the full-length heavy chain and/or full-length light chain nucleotide sequences may be 60%, 70%, 80%, 90%, 95%, 96%, 97%, 98% or 99% homologous to the sequences of interest.

In other embodiments, the variable regions of heavy chain and/or light chain nucleotide sequences may be 60%, 70%, 80%, 90%, 95%, 96%, 97%, 98% or 99% homologous to the sequences set forth above. An antibody having a variable region heavy chain and light chain having high (i.e., 80% or greater) homology to the variable region heavy chains and variable region light chains of a sequences encoding them, can be obtained by mutagenesis (e.g., site-directed or PCR-mediated mutagenesis) of nucleic acid molecules encoding the heavy and light chains, followed by testing of the encoded altered antibody for retained function (i.e., the functions set forth above) using the functional assays described herein.

As used herein, the percent homology between two amino acid sequences or two nucleotide sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al., 1990 J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., 1997 Nucleic Acids Res. 25(17):3389-3402. When utilizing BLAST and Gapped BLAST programs, the default parameters of the respective programs (e.g., XBLAST and NBLAST) can be used. See http:Ilwww.ncbi.nhn.nih.gov.

Antibodies with Conservative Modifications

In certain embodiments, an antibody of the invention has a heavy chain variable region consisting of CDR1, CDR2, and CDR3 sequences and a light chain variable region consisting of CDR1, CDR2, and CDR3 sequences, wherein one or more of these CDR sequences have specified amino acid sequences based on the antibodies described herein or conservative modifications of these, and wherein the antibodies retain the desired functional properties of the anti-target antibodies of the invention. Accordingly, the invention provides an isolated monoclonal antibody, or antigen binding portion of these, consisting of a heavy chain variable region consisting of CDR1, CDR2, and CDR3 sequences and a light chain variable region consisting of CDR1, CDR2, and CDR3 sequences, wherein: the heavy chain variable regions of CDR1 is sequences consisting of amino acid sequences, and conservative modifications of these; the heavy chain variable region of CDR2 is sequences consisting of amino acid sequences selected from the group consisting of amino acid sequences, and conservative modifications of these; the heavy chain variable region of CDR3 is sequences consisting of amino acid sequences selected from the group consisting of amino acid sequences, and conservative modifications of these; the light chain variable regions of CDR1 is sequences consisting of amino acid sequences, and conservative modifications of these; the light chain variable regions of CDR2 is sequences consisting of amino acid sequences selected from the, and conservative modifications of these; the light chain variable regions of CDR3 is sequences consisting of amino acid sequences, and conservative modifications of these; the antibody specifically binds to the target; and the antibody exhibits at least one functional properties: the binds target.

In various embodiments, the antibody may exhibit one or more, two or more, or three or more of the functional properties listed discussed above. Such antibodies can be, for example, human antibodies, humanized antibodies or chimeric antibodies.

In various embodiments, the antibody may exhibit one or more, two or more, or three or more of the functional properties. Such antibodies can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family, and the altered antibody can be tested for retained function using the functional assays described herein.

Further, the conservative amino acid substitutions can be made in any of the epitopes above, and are within the scope of the invention herein. For example in SEQ ID NO: 5 provided herein having an amino acid sequence EDLS, the amino acids E and D can be interchanged, so that DDLS (SEQ ID NO: 21) and EELS (SEQ ID NO: 22) are within the scope of the invention.

Antibodies that Bind to the Same Epitope

In another embodiment, the invention provides one, or a combination of antibodies, each of which binds to the same epitope as do the various anti-target antibodies of the invention provided herein, or a combination of such an antibody with a different antibody having a different binding specificity. Such additional antibodies can be identified based on their ability to cross-compete (e.g., to competitively inhibit the binding of, in a statistically significant manner) with other antibodies of the invention in standard target binding assays. The ability of a test antibody to inhibit the binding of antibodies of the present invention to human target demonstrates that the test antibody can compete with that antibody for binding to target; such an antibody may, according to non-limiting theory, bind to the same or a related (e.g., a structurally similar or spatially proximal) epitope on human target as the antibody with which it competes. In a certain embodiment, the antibody that binds to the same epitope on human target as the antibodies of the present invention is a human monoclonal antibody.

Engineered and Modified Antibodies

An antibody of the invention further can be prepared using an antibody having one or more of the V_{H} and/or V_{L} sequences shown herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad. See. U.S.A. 86:10029-10033; U.S. Pat. No. 5,225,539 to winter, and U.S. Pat. Nos. 5,530,101; 5,585,089; 5,693,762 and 6,180,370 to Queen et al.)

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836; the contents of each of which are expressly incorporated herein by reference.

An example of framework sequences for use in the antibodies of the invention are those that are structurally similar to the framework sequences used by selected antibodies of the invention, e.g., consensus sequences and/or framework sequences used by monoclonal antibodies of the invention. The V_{H} CDR1, 2 and 3 sequences, and the V_{L} CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody. In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired staphylococcal protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in PCT Publication WO 00/42072 by Presta. Moreover, the binding sites on human IgG1 for FcγR1, FcγRII, FcγRIII and FcRn have been mapped and variants with improved binding have been described (see Shields, R. L. et al., 2001 J. Biol. Chen. 276:6591-6604).

In still another embodiment, the glycosylation of an antibody is modified. For example, an aglycoslated antibody can be made (i.e., the antibody lacks glycosylation). Glycosylation can be altered to, for example, increase the affinity of the antibody for “antigen”. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment of these, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

Methods of Engineering Antibodies

As discussed above, anti-target antibodies having V_{H} and V_{L} sequences or full-length heavy and light chain sequences shown herein can be used to create new anti-target antibodies by modifying full-length heavy chain and/or light chain sequences, V_{H} and/or V_{L} sequences, or the constant region(s) attached thereto. Thus, in another aspect of the invention, the structural features of an anti-target antibody of the invention are used to create structurally related anti-target antibodies that retain at least one functional property of the antibodies of the invention, such as binding to human target and also inhibiting one or more functional properties of the target (e.g., recognizing and binding to the cartilage breakdown peptide as described herein).

For example, one or more CDR regions of the antibodies of the present invention, or mutations of these, can be combined recombinantly with known framework regions and/or other CDRs to create additional, recombinantly-engineered, anti-target antibodies of the invention, as discussed above. Other types of modifications include those described in the previous section. The starting material for the engineering method is one or more of the V_{H} and/or V_{L} sequences provided herein, or one or more CDR regions thereof. To create the engineered antibody, it is not necessary to actually prepare (i.e., express as a protein) an antibody having one or more of the V_{H} and/or V_{L} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Standard molecular biology techniques can be used to prepare and express the altered antibody sequence. The antibody encoded by the altered antibody sequence(s) is one that retains one, some or all of the functional properties of the anti-target antibodies described herein, which functional properties include, but are not limited to, specifically binding to human target; and the antibody exhibits at least one of the following functional properties: the antibody inhibits binding of target to a target receptor, or the antibody inhibits a target receptor binding thereby preventing progression of a disease state.

The functional properties of the altered antibodies can be assessed using standard assays available in the art and/or described herein, as set forth herein (e.g., ELISAs). In certain embodiments of the methods of engineering antibodies of the invention, mutations can be introduced randomly or selectively along all or part of an anti-target antibody coding sequence and the resulting modified anti-target antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

Nucleic Acid Molecules Encoding Antibodies of the Invention

Another aspect of the invention pertains to nucleic acid molecules that encode the antibodies of the invention. The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. A nucleic acid of the invention can be, for example, DNA or RNA and may or may not contain intronic sequences. In an embodiment, the nucleic acid is a cDNA molecule. The nucleic acid may be present in a vector such as a phage display vector, or in a recombinant plasmid vector.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from various phage clones that are members of the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat, E. A., et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as to a Fab light chain gene) by operatively linking the V_{L}-encoding DNA to another DNA molecule encoding the light chain constant region, CL. The sequences of human light chain constant region genes are known in the art (see e.g., Kabat, E. A., et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or a lambda constant region.

To create an scFv gene, the V_{H}- and V_{L}-encoding DNA fragments are operatively linked to another fragment encoding a flexible linker, e.g., encoding the amino acid sequence (Gly4-Ser)₃, such that the V_{H} and V_{L} sequences can be expressed as a contiguous single-chain protein, with the V_{L} and V_{H} regions joined by the flexible linker (see e.g., Bird et al., 1988 Science 242:423-426; Huston et al., 1988 Proc. Natl. Acad. Sci. USA 85:5879-5883; McCafferty et al., 1990 Nature 348:552-554).

Production of Monoclonal Antibodies of the Invention

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.

In a certain embodiment, the antibodies of the invention are human monoclonal antibodies. Such human monoclonal antibodies directed against the target can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N. Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise anti-target antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-target antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise anti-target antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

The mouse splenocytes, isolated from the HuMab mice and KM mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the number of SP2/0 nonsecreting mouse myeloma cells (ATCC, CRL 1581) with 50% PEG (Sigma). Cells are plated at approximately 1×10⁵/well in flat bottom microtiter plate, followed by about two week incubation in selective medium containing 10% fetal bovine serum, 10% P388D 1(ATCC, CRL TIB-63) conditioned medium, 3-5% origen (IGEN) in DMEM (Mediatech, CRL 10013, with high glucose, L-glutamine and sodium pyruvate) plus 5 mM HEPES, 0.055 mM 2-mercaptoethanol, 50 mglnni gentamycin and 1× HAT (Sigma, CRL P-7185). After 1-2 weeks, cells are cultured in medium in which the HAT is replaced with HT. Individual wells are then screened by ELISA for human anti-target monoclonal IgG antibodies. Once extensive hybridoma growth occurred, medium is monitored usually after 10-14 days. The antibody secreting hybridomas are replated, screened again and, if still positive for human IgG, anti-target monoclonal antibodies are subcloned at least twice by limiting dilution. The stable subclones are then cultured in vitro to generate small amounts of antibody in tissue culture medium for further characterization.

Immunization of Human Ig Mice

When human Ig mice are used to raise human antibodies of the invention, such mice can be immunized with a purified or enriched preparation of the target antigen and/or recombinant target, or target fusion protein, as described by Lonberg, N. et al., 1994 Nature 368(6474): 856-859; Fishwild, D. et al., 1996 Nature Biotechnology 14: 845-851; and PCT Publication WO 98124884 and WO 01/14424. The mice can be 6-16 weeks of age upon the first infusion. For example, a purified or recombinant preparation (5-50 μg) of the target antigen can be used to immunize the human Ig mice intraperitoneally. Alternatively as shown herein, a small oligopeptide as shown in SEQ ID NO: 1 that contains the antigenic determinant DSL is used to immunize animals such as mice.

Many of the standard procedures are shared by those used for injecting wild type or non-recombinant mice. Detailed procedures to generate fully human monoclonal antibodies to the target are described above. Cumulative experience with various antigens has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA, and mice with sufficient titers of anti-target human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo12).

Generation of Hybridomas Producing Human Monoclonal Antibodies

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×145 in flat bottom microtiter plates, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0:055 mM 2-mercaptoethanol, 50 units/ml penicillin, 50 mg/ml streptomycin, 50 mg/ml gentamycin and 1× HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −800° C.

Generation of Transfectomas Producing Monoclonal Antibodies

Antibodies of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. 1990). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe, Y. et al., 1988 Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. patents with U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the invention in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R., 1985 Immunology Today 6:12-13).

Mammalian host cells for expressing the recombinant antibodies of the invention include Chinese Hamster Ovary (CHO cells) (including dhfr-CHO cells, described Urlaub and Chasin, 1980 Proc. Natl. Acad. Sci. USA 77:4216-4220 used with a DH FR selectable marker, e.g., as described in R. J. Kaufman and P. A. Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

Immunoconjugates

In another aspect, the present invention features an anti-target antibody, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxon, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), ablating agents (e.g., mechlorethamine, thioepa chloraxnbucil, meiphalan, carmustine (BSNU) and lomustine (CCNU), cyclothosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg™; Wyeth-Ayerst).

Cytoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al., 2003 Adv. Drug Deliv. Rev. 55:199-215; Trail, P. A. et al., 2003 Cancer Immunol. Immunother. 52:328-337; Payne, G., 2003 Cancer Cell 3:207-212; Allen, T. M., 2002 Nat. Rev. Cancer 2:750-763; Pastan, I. and Kreitman, R. J., 2002 Curr. Opin. Investig. Drugs 3:1089-1091; Senter, P. D. and Springer, C. J., 2001 Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, indium¹¹¹, yttrium⁹⁰, and lutetium⁷⁷. Method for preparing radioimmunconjugates are established in the art. Examples of radioimmunoconjugates are commercially available, including Zevalin™ (DEC Pharmaceuticals) and Bexxar™ (Corixa Pharmaceuticals), and similar methods can be used to prepare radioimmunoconjugates using the antibodies of the invention.

The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Bispecific Molecules

In another aspect, the present invention features bispecific molecules comprising an anti-target antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for the target and a second binding specificity for a second target epitope. For example, the second target epitope is an Fc receptor, e.g., human FcγR1 (CD64) or a human Fcα receptor (CD89). Therefore, the invention includes bispecific molecules capable of binding both to FcγR, FcαR or FcεR expressing effector cells (e.g., monocytes, macrophages or polymorphonuclear cells (PMNs), and to target cells expressing the target. These bispecific molecules target the target expressing cells to effector cell and trigger Fc receptor-mediated effector cell activities, such as phagocytosis of an the target expressing cells, antibody dependent cell-mediated cytotoxicity (ADCC), cytokine release, or generation of superoxide anion.

Additionally, for the invention in which the bispecific molecule is multi-specific, the molecule can further include a third binding specificity, in addition to an anti-Fc binding specificity and an anti-target binding specificity. For example, the third binding specificity could be an anti-enhancement factor (EF) portion, e.g., a molecule that binds to a surface protein involved in cytotoxic activity and thereby increases the immune response against the target cell. The “anti-enhancement factor portion” could be an antibody, functional antibody fragment or a ligand that binds to a given molecule, e.g., an antigen or a receptor, and thereby results in an enhancement of the effect of the binding determinants for the Fc receptor or target cell antigen.

The “anti-enhancement factor portion” can bind an Fc receptor or a target cell antigen. Alternatively, the anti-enhancement factor portion could bind to an entity that is different from the entity to which the first and second binding specificities bind. For example, the anti-enhancement factor portion can bind a cytotoxic T-cell (e.g. by CD2, CD3, CD8, CD28, CD4, CD44, ICAM-1 or other immune cell that results in an increased immune response against the target cell).

In one embodiment, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fγ receptor classes: FcγR1 (CD64), FcγRII(CD32), and FcγRIII (CD 16). In another embodiment, the Fcγ receptor is a human high affinity FcγRI. The human FcγRI is a 72 kDa molecule, which shows high affinity for monomeric IgG (10⁸-10⁹ M⁻¹).

The production and characterization of certain anti-Fcγ monoclonal antibodies are described by Fanger et al. in PCT Publication WO 88/00052 and in U.S. Pat. No. 4,954,617, the teachings of which are fully incorporated by reference herein. These antibodies bind to an epitope of FcγRI, FcγRII or FcγRIII at a site that is distinct from the Fcγ binding site of the receptor and, thus, binding of antibodies is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al., 1995 J. Immunol 155 (10): 4996-5002 and PCT Publication WO 94/10332. The 1122 antibody producing cell line was deposited at the American Type Culture Collection under the designation HA022CL1 and has the accession no. CRL 11177.

In still other embodiments, the binding specificity for an Fc receptor is provided by an antibody that binds to a human IgA receptor, e.g., an Fc-alpha receptor (FcαRI (CD89), the binding of which does not have to be blocked by human immunoglobulin A (IgA). The term “IgA receptor” is intended to include the gene product of one a gene (FcαRI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has an intermediate or medium affinity (5×10⁷ M⁻¹) for both IgA1 and IgA2, which is increased upon exposure to cytokines such as G-CSF or GM-CSF (Morton, H. C. et al., 1996 Critical Reviews in Immunology 116:423-440). Four FcαRI-specific monoclonal antibodies, identified as A3, A59, A62 and A77, which bind FcαRI outside the IgA ligand binding domain, have been described (Monteiro, R. C. et al., 1992 J. Immunol. 148:1764).

FcαRI and FcγRI are trigger receptors for use in the bispecific molecules of the invention because they are expressed primarily on immune effector cells, e.g., monocytes, PMNs, macrophages and dendritic cells; expressed at high levels (e.g., 5,000-100,000 per cell); mediators of cytotoxic activities (e.g., ADCC, phagocytosis); mediate enhanced antigen presentation of antigens, including self-antigens, targeted to them.

Other antibodies which can be employed in the bispecific molecules of the invention are murine, chimeric and humanized monoclonal antibodies.

The bispecific molecules of the present invention can be prepared by conjugating the constituent binding specificities, e.g., the anti-FcR and anti-target binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78,118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. patents having U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively 4 labeled and used in a radioimmunoassay (RIA) (see, for example, Weintraub; B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

Pharmaceutical Compositions

In another aspect, the present invention provides a composition, e.g., a pharmaceutical composition, containing one or a combination of polyclonal antibodies, or one or a combination of monoclonal antibodies, or one or a combination of a polyclonal antibody and a monoclonal antibody, or antigen-binding portion(s) thereof, of the present invention, formulated together with a pharmaceutically acceptable carrier. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecifics) that bind to different epitopes on the target antigen or that have complementary activities.

Pharmaceutical compositions of the invention also can be administered in combination therapy, i.e., combined with other agents. For example, the combination therapy can include an anti-target antibody of the present invention combined with at least one other anti-inflammatory or anti-proliferative agent. Examples of therapeutic agents that can be used in combination therapy are described in greater detail below in the section on uses of the antibodies of the invention.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound, i.e., antibody, immunoconjuage, or bispecific molecule, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts, and/or gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 per cent to about ninety-nine percent of active ingredient, from about 0.1 per cent to about 70 per cent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Dosage regimens for an anti-target antibody of the invention include 1 mg/kg body weight or 3 mg/kg body weight by intravenous administration, with the antibody being given using one of the following dosing schedules: every four weeks for six dosages, then every three months; every three weeks; 3 mg/kg body weight once followed by 1 mg/kg body weight every three weeks.

In some methods, a combination having two or more monoclonal antibodies or polyclonal antibodies, each antibody having a different binding specificity, is administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-target antibody of the invention can results in a decrease in severity of disease symptoms, an increase in frequency and duration of disease symptom-free periods, or a prevention of impairment or disability due to the disease affliction.

A composition of the present invention can be administered by one or more routes of administration using one or more of a variety of methods known in the art. As will be appreciated by the skilled artisan, the route and/or mode of administration will vary depending upon the desired results. Routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion, and particularly includes injection or infusion directly into ajoint. Alternatively, an antibody of the invention is administered by a nonparenteral route, such as a topical, epidermal or mucosal route of administration, for example, intranasal, oral, vaginal, rectal, sublingual or topical.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. patents having U.S. Pat. Nos.: 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chemother. 39:180); surfactant protein A receptor (Briscoe et al., 1995 Am. J. Physiol.1233:134); p120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

Uses and Methods of the Invention

The antibodies (and immunoconjugates and bispecific molecules) of the present invention have in vitro and in vivo diagnostic, prognostic and therapeutic utilities. For example, these molecules can be administered to cells in culture, e.g. in vitro or in vivo, or in a subject, e.g., in vivo, to treat, prevent or diagnose a variety of disorders. The antibodies are incorporated into kits for diagnosis of various body fluids, most particularly, blood and blood products such as serum, or urine, and most particularly, synovial fluid.

The term “subject” as used herein in intended to include human and non-human animals. Non-human animals includes all vertebrates, e.g., mammals and non-mammals, such as non-human primates, sheep, dogs, cats, cows, horses, chickens, amphibians, and reptiles. The methods are particularly suitable for treating human patients having a disorder associated with aberrant the target expression. When antibodies to the target are administered together with another agent, the two can be administered in either order or simultaneously.

In one embodiment, the antibodies (and immunoconjugates and bispecific molecules) of the invention can be used to detect levels of the target, or levels of cells that contain the target. This can be achieved, for example, by contacting a sample (such as an in vitro sample) and a control sample with the anti-target antibody under conditions that allow for the formation of a complex between the antibody and the target. Any complexes formed between the antibody and the target are detected and compared in the sample and the control. For example, standard detection methods, well known in the art, such as ELISA and flow cytometic assays, can be performed using the compositions of the invention.

Accordingly, in one aspect, the invention further provides methods for detecting the presence of the target peptide ligand (e.g., the human target antigen) in a sample, or for measuring the amount of the target, the method including contacting the sample, and a control sample, with an antibody of the invention, or an antigen binding portion thereof, which specifically binds to the target, under conditions that allow for formation of a complex between the antibody or portion thereof and the target. The formation of a complex is then detected, and a difference in complex formation between the sample compared to the control sample indicates the presence of the target in the sample. Further, the extent of the presence of the target indicates extent of cartilage degradation, i.e., the stage of progression of an arthritic condition.

Also within the scope of the invention are kits including the compositions (e.g., antibodies, human antibodies, immunoconjugates and bispecific molecules) of the invention and instructions for use. The kit can further contain a least one additional reagent, or one or more additional antibodies of the invention (e.g., an antibody having a complementary activity which binds to an epitope on the target antigen distinct from the first antibody) packaged individually or in combination. Kits typically include a label indicating the intended use of the contents of the kit. The term label includes any writing, or recorded material supplied on or with the kit, or which otherwise accompanies the kit. The kit includes a container.

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

### EXAMPLES

### Example 1

### Preparation of Antibody Specific for a Cartilage Aggrecan Cleavage Site

Aggrecanase attacks the stable intercellular matrix and generates both Ala393-Glu1564 and the “target” Ala393-Ser1411. The specific target is generated by aggrecanase-mediated cleavage of a pre-existing calpain product (see FIG. 1). The sequences of the junctions of the target have been determined herein, and these sequences used to develop an antibody that is specific for the epitope created by digestion by aggrecanase.

Antibody was produced by immunizing an animal, by contacting it with a synthetic peptide having amino acid sequence cys-gly-gly-ser-gly-val-glu-asp-leu-ser (SEQ ID NO: 1). Data showed that the antibody obtained recognizes the target peptide, i.e., is a ligand of this target.

The antibody was obtained from immunized rabbits, is identified herein as JSCDLS, and was used to coat wells of a multi-well dish, at various dilutions. To serve a positive control for antibody binding, the target was produced by preparing aggrecanase digests of human mature aggrecan (prepared according as shown in Sandy et al., Biochem J 2001 Sep. 15 358: 615-626; see band “f”). The amount of target was determined by analysis using polyacrylamide gel electrophoresis, and the target preparation was diluted in order to generate a standard curve for analysis of clinical samples. The buffer diluent serves as a negative control, for the amount of antibody in each dilution.

### Example 2

### Correlating Target with Arthritic Conditions

Samples of body fluids are obtained from human subjects, in two groups: patients suffering with arthritic conditions, and normal subjects, are screened along with the dilutions of the target. In broad outline, volumes of each sample dilution are added to separate wells of the multi-well plate previously coated with JSCDLS antibody. Target molecules carrying the DLS epitope are immobilized to the antibody coated to the wells, and the wells are evacuated so that remaining volume of sample with unbound material is discarded, and the wells are washed. The target bound to the antibody is detected calorimetrically by one of numerous methods known to those of skill in the art of immunology (see Harlow, E. et al., 1988, Antibodies: a Laboratory Manual, Cold Spring Harbor Press, Cold Spring Harbor, N.Y.).

Samples from arthritic patients are found to contain significantly larger amounts of target, as determined by binding to the immobilized JSCDLS antibody.

### Example 3

### Pretreatment of Patient Samples Prior to Antibody Binding

In practice, it is convenient to pre-treat the samples, e.g., to trypsinize, and to digest with chondroitinase and neuraminidase, the target-containing fluids (to make disaccharide-substituted Ileu925-Ser1411). Samples are then reacted in wells of a multi-well dish, each well having been coated with antibody JSCDLS, to bind resulting further digested target Ileu925-Ser1411 to the antibody and then detect the “sandwich” peptide with peanut agglutinin peroxidase.

By any of the above techniques, target is assayed in various body fluids, most conveniently and least invasively being urine, and in synovial fluids.

### Example 4

### Detection of Anti-target Auto-antibodies Associated with RA

Rheumatoid arthritis (RA) is an auto-immune disease in which the patient elaborates antibodies against peptides found in cartilage. While a number of auto-antigens found in humans have been proposed to correlate with RA such as amino acid sequences in type II collagen, cyclic citrullinated peptides derived from keratin, filaggrin, glucose-6-phosphate isomerase, calpastatin, calreticulin and RA33, additional candidate peptides are discovered, such as peptidylarginine deiminase 4 (PADI4; see Takizawa et al., Scan J Rheumatol 2005 34(3): 212-215.

The target ligand of antibody JSCDLS described herein, which target is a peptide of aggrecan, thus may be found in complex with an auto-antibody in a sample of a body fluid from a subject having an autoimmune disease such as RA. To identify the presence of such autoantibodies, samples from arthritic patients are reacted with JSCDLS in plates coated as above with JSCDLS. That antibody, prepared from a non-human animal such as a rabbit, is coated onto multi-well dishes, to be used to immobilize the target peptide ligand found in human biological samples. Following incubation of samples in the wells of the dishes, the unbound fraction of each sample is removed, and the plates are rinsed as above. Any antibodies remaining after the rinse are specifically. bound to the target peptide.

Human antibody that is identified as present in the wells which is associated in some of the samples with an autoimmune reaction against the target peptide, is observed to be bound to the target peptide, and following the initial binding step, is then detected by using anti-human antibodies prepared in yet another (second) non-human animal such as a goat. The goat antibody is labeled with a detective marker, such as a fluorescent or enzymatic tag, as described in Harlow et al. Alternatively, the plate is initially coated with a derivative of antibody JSCDLS that has been converted to a Fab, i.e., an antibody fragment described above that carries the antigen-binding determinant portions of the antibody H and L chains and not the Fc portion. After reacting patient samples in wells coated with JSCDLS Fab in the step that immobilizes the target peptide, detection of human auto-antibodies bound to target is then achieved with an antibody that specifically recognizes and binds to amino acid sequences specific for human antibodies. Alternatively, detection of human auto-antibodies bound to target is then achieved with an antibody that specifically recognizes and binds to Fc sequences.

What is claimed is:

1. An antibody that binds to a target ligand comprising a calpain-generated epitope, wherein aggrecanase-mediated degradation of aggrecan releases the target from cartilage in a tissue of a subject.

2. The antibody according to claim 1, wherein the calpain is m-calpain.

3. The antibody according to claim 1, wherein the target has a carboxy-terminal amino acid sequence comprising aspartic acid-leucine-serine (DLS).

4. The antibody according to claim 1, wherein the ligand is soluble in body fluids.

5. The antibody according to claim 1, wherein the target has an amino-terminal comprising the sequence alanine-arginine-glycine (ARG).

6. The antibody according to claim 1 selected from at least one of the group of antibodies consisting of a polyclonal, monoclonal, Fab, single chain antibody (scAb) and an antigen-binding determinant of a heavy chain and a light chain.

7. The antibody according to claim 6, wherein the antibody is polyclonal.

8. The antibody according to claim 6, wherein the antibody is an IgG.

9. The antibody according to claim 3, wherein target detected in a sample of body fluid is human aggrecan peptide Ala393-Ser1411.

10. The antibody according to claim 7, produced by contacting an animal with a peptide having an amino acid sequence selected from at least one of the group: cys-gly-gly-ser-gly-val-glu-asp-leu-ser (SEQ ID NO: 1), glu-asp-leu-ser (SEQ ID NO: 5), val-glu-asp-leu-ser (SEQ ID NO: 6), gly-val-glu-asp-leu-ser (SEQ ID NO: 7), ser-gly-val-glu-asp-leu-ser (SEQ ID NO: 8), gly-ser-gly-val-glu-asp-leu-ser (SEQ ID NO: 9), and gly-gly-ser-gly-val-glu-asp-leu-ser (SEQ ID NO: 10), and conservative variants or fragments thereof.

11. The antibody according to claim 1, wherein the target comprises a degradation product of stable matrix aggrecan.

12. The antibody according to claim 1, produced by contacting an animal with a peptide having an amino acid sequence selected from at least one of the group: arg-leu-pro-ser-gly-glu-glu (SEQ ID NO: 2), arg-leu-pro-ser (SEQ ID NO: 11), arg-leu-pro-ser-gly (SEQ ID NO: 12), and arg-leu-pro-ser-gly-glu (SEQ ID NO: 13), and conservative variants or fragments thereof.

13. A composition comprising two or more antibodies according to claim 1.

14. A method of making the antibody according to claim 1 comprising:
contacting an animal with a synthetic peptide having an amino acid sequence that is an calpain substrate, wherein the animal produces the antibody; and
obtaining the antibody from the serum of the animal.

15. The method according to claim 14, wherein the animal is selected from at least one of the group of rabbit; mouse; rat; dog; horse; cow; sheep; pig; and goat.

16. The method according to claim 14, wherein the amino acid sequence comprises a sequence selected from at least one of the group consisting of asp-leu-ser (DLS), arg-leu-pro (RLP), and conservative variants thereof.

17. A method of assaying cartilage destruction in a subject comprising
contacting a sample of a body fluid of the subject with at least one antibody according to claim 1; and
determining a presence of at least one epitope comprising amino acid sequences aspartic acid leucine-serine (DLS) or arg-leu-pro (RLP) in the body fluid, wherein the sequence binds to the antibody, and the amount of the epitope is a measure of cartilage destruction in the subject.

18. The method according to claim 17, wherein the subject is at risk for having an arthritic condition.

19. The method according to claim 18, wherein the arthritic condition is selected from at least one of rheumatoid arthritis, osteoarthritis, juvenile arthritis, traumatic injury, ankylosing spondylitis, infectious arthritis, psoriatic arthritis, lumbosacral arthritis, systemic lupus erythematosus (SLE), degenerative disc disease, gout, pseudogout, and reactive arthritis.

20. The method according to claim 17, wherein the antibody is a polyclonal antibody.

21. The method according to claim 17, wherein the antibody is a monoclonal antibody.

22. The method according to claim 17, wherein the antibody is immobilized.

23. The method according to claim 17, wherein the epitope is detectably labeled.

24. The method according to claim 23, wherein the labeled epitope is at least one of selected from radioactive, chemiluminescent, bioluminescent, and calorimetric.

25. The method according to claim 23, further comprising prior to determining, biotinylating the epitope.

26. The method according to claim 17, further comprising prior to determining, treating the sample with trypsin, chondroitinase and neuraminidase and detecting a resulting disaccharide-substituted Ileu925-Ser1411 derivative of the target.

27. The method according to claim 17, further comprising prior to determining, deglycosylating the sample with an enzyme selected from at least one of the group chondroitinase ABC, chondroitinase A, chondroitinase B, chondroitinase C, chondroitinase AC, keratanase, and keratanase II.

28. The method according to claim 26, further comprising contacting a complex of antibody and resulting derivative of the target with peanut agglutinin peroxidase.

29. The method according to claim 17, further comprising prior to contacting, providing the sample from the subject selected from at least one of the group consisting of blood, urine, synovial fluid, tears, sweat, saliva, serum, lymph, semen, vaginal fluid, cerebro-spinal fluid, cell culture supernatant, cell extract, and tissue extract.

30. The method according to claim 17, further comprising prior to contacting, providing the sample from the subject selected from at least one of the group consisting of urine, synovial fluid, serum, and tissue extract.

31. The method according to claim 17, wherein the at least one antibody is immobilized on a support substrate selected from at least one of the group of a bead, a slide, a gel, a multi-well plate, and a column.

32. The method according to claim 17, further comprising comparing the amount of the epitopes in the sample to a control lacking the epitopes.

33. The method according to claim 17, further comprising prior to contacting, administering to the subject an agent for treating a cartilage destruction condition, wherein the amount of epitope is a measure of prevention of progressive cartilage destruction.

34. A method of assaying for prevention or amelioration of progressive cartilage destruction in a subject comprising
administering to the subject an agent for treating a cartilage destruction condition;
contacting at least one sample of a body fluid of the subject with at least one antibody according to claim 1; and
determining a presence of at least one epitope comprising amino acid sequences aspartic acid leucine-serine (DLS) or arg-leu-pro (RLP) in the fluid, wherein the epitope binds to the antibody, and the amount of the epitope that binds is a measure of cartilage destruction or prevention of destruction in the subject, compared to a control sample of the body fluid obtained prior to administering, or to a control subject not administered the agent.

35. A method for assaying activity of m-calpain in a sample in need of an assay, the method comprising:
contacting the sample with an m-calpain substrate to produce a resulting reaction mix, wherein the substrate is aggrecan, or a synthetic peptide having an amino acid sequence of an m-calpain digestion site; and
determining a presence and an amount of m-calpain, by reacting the the reaction mix with at least one antibody specific for a neo-epitope resulting from m-calpain digestion of the aggrecan, wherein the extent of the reaction of the sample with the at least one antibody, in comparison to a control reaction in the absence of the sample, is an indication of the presence and the amount of m-calpain.

36. The method according to claim 35, wherein the synthetic peptide comprises an amino acid sequence asp-leu-ser-arg-leu-pro (DLSRLP; SEQ ID NO: 14).

37. The method according to claim 36, wherein the synthetic peptide comprises an amino acid sequence selected from at least one of the group: asp-leu-ser-arg-leu-pro-ser (DLSRLPS; SEQ ID NO: 15); glu-asp-leu-ser-arg-leu-pro (EDLSRLP; SEQ ID NO: 16); asp-leu-ser-arg-leu-pro-ser-gly (DLSRLPSG; SEQ ID NO: 17); val-glu-asp-leu-ser-arg-leu-pro (VEDLSRLP; SEQ ID NO: 18); asp-leu-ser-arg-leu-pro-ser-gly-glu (DLSRLPSGE; SEQ ID NO: 19); and gly-val-glu-asp-leu-ser-arg-leu-pro (GVEDLSRLP; SEQ ID NO: 20).

38. A method for detecting in a sample of a body fluid from a subject, an autoantibody bound to an aggrecan peptide, the method comprising:
immobilizing on a solid surface at least one antibody produced by immunizing an animal with a synthetic peptide having amino acid sequence as shown in any of SEQ ID NOs: 1-13;
contacting the surface with the fluid, wherein the aggrecan peptide and autoantibody bind to the surface; and
detecting the autoantibody with a reagent that binds to at least one Fc constant amino acid sequence for the subject species, wherein the autoantibody is detected in the sample.

39. The method according to claim 38, wherein the subject is a human.

40. The method according to claim 39, wherein the subject is identified as having or being at risk for an autoimmune disease.

41. The method according to claim 40, wherein the autoimmune disease is rheumatoid arthritis.

42. A kit comprising an antibody according to claim 1 for assaying cartilage destruction in a subject or effectiveness of a treatment to ameliorate or prevent progressive cartilage destruction, wherein the antibody is present in a unit dose.

43. The kit according to claim 42, wherein the antibody is immobilized in a plurality of wells in a multi-well plate.

44. The kit according to claim 42, further comprising instructions for use.

45. The kit according to claim 42, further comprising a positive control.

46. The kit according to claim 42, wherein the positive control is an amount of the aggrecan target ligand.
